(19) United States
(12) Patent Application Publication
(10) Pub. No.: US 2024/0000312 A1
(43) Pub. Date: Jan. 4, 2024

(54) System and Method for Monitoring and Responding to Surrounding Context

(71) Applicant: Apple Inc., Cupertino, CA (US)

(72) Inventors: Jean-Charles Bernard Marcel Bazin, Sunnyvale, CA (US); Anselm Grundhoefer, Campbell, CA (US); Arun Srivatsan Rangaprasad, Sunnyvale, CA (US); Giuliano Pasqualotto, Los Altos, CA (US)

(21) Appl. No.: 18/344,468

(22) Filed: Jun. 29, 2023

Related U.S. Application Data
(60) Provisional application No. 63/367,261, filed on Jun. 29, 2022.

(51) Int. Cl. A61B 3/14; A61B 3/113

(52) U.S. Cl. CPC A61B 3/14; A61B 3/113

(57) ABSTRACT

Performing a corrective operation for environmental conditions related to a predetermined eye condition includes obtaining environment sensor data from a one or more sensors of the device, determining a current context for the device based on the environment sensor data, and determining, based on the current context, that an eye state criterion is satisfied. In response to determining that the eye state criterion is satisfied, a corrective operation is determined in accordance with the eye state criterion, and the corrective operation is performed. When performed, the corrective operation is configured to resolve an environmental condition associated with the eye state criterion.

### FIELD OF THE INVENTION

This disclosure relates generally to image processing. More particularly, but not by way of limitation, this disclosure relates to techniques and systems for monitoring a physical environment and performing corrective operations in response to the context in the physical environment.

### BACKGROUND

Certain activities can affect an eye state of a user's eyes if performed in a particular environment. For example, watching screens in a dark room, reading in the dark, and the like may cause a state change in the user's eyes. Further, some user activity may affect an eye state based on how long the user is performing the activity, such as driving for a certain period of time or staring at an object for a certain period of time, such as computer screens, knitting, etc. For example, eyes can experience redness, dryness (e.g., from infrequent blinking), vergence (i.e., looking at something too close), and the like.

### BRIEF DESCRIPTION OF THE DRAWINGS

FIG. 1 shows a diagram of an environment in which variations of the disclosure are utilized, according to one or more embodiments.

FIG. 2 shows a flow diagram of a technique for managing an operation of a device to address an eye state, according to one or more embodiments.

FIG. 3 shows, in flowchart form, an example process for performing a corrective operation based on an eye status, in accordance with one or more embodiments.

FIG. 4 shows, in flowchart form, an example process for performing a corrective action based on a predicted eye status, in accordance with one or more embodiments.

FIG. 5 shows, in block diagram form, an example network diagram, according to one or more embodiments.

FIG. 6 shows, in block diagram form, a mobile device in accordance with one or more embodiments.

### DETAILED DESCRIPTION

In general, embodiments described herein are directed to a technique for adjusting an operation of a device in response to detected environmental conditions. In some embodiments, scenarios or activities are automatically detected by a system which might have an impact on user experience, for example by affecting eye state. Then the system may cause a change in one or more environmental conditions to adjust the impact on the user experience.

In some embodiments, sensors embedded in a portable device may collect environmental sensor data from which an environmental condition may be determined. Further, in some embodiments, a user activity may be determined, even if the user activity is not an activity provided by the portable device. For example, the portable device includes an optical see-through display. Thus, a user may be engaged in activities that involve, or do not involve, the portable device at a given time. Thus, the portable device may determine, or predict, a user activity based on state information of the portable device, such as apps running and the like, and/or from external indications, such as environmental sensor data, detected user cues or other indications detectable by components of the portable device. The portable device may have the ability to modify the environmental conditions for the user, for example by turning on a light in a darkened room, adjusting display settings of the portable device and/or a remote device, activating a filter, and the like.

In the following description, for purposes of explanation, numerous specific details are set forth in order to provide a thorough understanding of the disclosed concepts. As part of this description, some of this disclosure's drawings represent structures and devices in block diagram form in order to avoid obscuring the novel aspects of the disclosed concepts. In the interest of clarity, not all features of an actual implementation may be described. Further, as part of this description, some of this disclosure's drawings may be provided in the form of flowcharts. The boxes in any particular flowchart may be presented in a particular order. It should be understood, however, that the particular sequence of any given flowchart is used only to exemplify one embodiment. In other embodiments, any of the various elements depicted in the flowchart may be deleted, or the illustrated sequence of operations may be performed in a different order, or even concurrently. In addition, other embodiments may include additional steps not depicted as part of the flowchart. Moreover, the language used in this disclosure has been principally selected for readability and instructional purposes and may not have been selected to delineate or circumscribe the inventive subject matter, it being necessary to resort to the claims in order to determine such inventive subject matter. Reference in this disclosure to “one embodiment” or to “an embodiment” means that a particular feature, structure, or characteristic described in connection with the embodiment is included in at least one embodiment of the disclosed subject matter, and multiple references to “one embodiment” or “an embodiment” should not necessarily be understood as all referring to the same embodiment.

It will be appreciated that, in the development of any actual implementation (as in any software and/or hardware development project), numerous decisions must be made to achieve a developer's specific goals (e.g., compliance with system- and business-related constraints) and that these goals may vary from one implementation to another. It will also be appreciated that such development efforts might be complex and time-consuming but would nevertheless be a routine undertaking for those of ordinary skill in the design and implementation of multi-modal processing systems having the benefit of this disclosure.

Various examples of electronic systems and techniques for using such systems in relation to various technologies are described.

A physical environment, as used herein, refers to a physical world that people can sense and/or interact with without aid of electronic devices. The physical environment may include physical features such as a physical surface or a physical object. For example, the physical environment corresponds to a physical park that includes physical trees, physical buildings, and physical people. People can directly sense and/or interact with the physical environment such as through sight, touch, hearing, taste, and smell. In contrast, an extended reality (XR) environment refers to a wholly or partially simulated environment that people sense and/or interact with via an electronic device. For example, the XR environment may include augmented reality (AR) content, mixed reality (MR) content, virtual reality (VR) content, and/or the like. With an XR system, a subset of a person's physical motions, or representations thereof, are tracked, and, in response, one or more characteristics of one or more virtual objects simulated in the XR environment are adjusted in a manner that comports with at least one law of physics. As one example, the XR system may detect head movement and, in response, adjust graphical content and an acoustic field presented to the person in a manner similar to how such views and sounds would change in a physical environment. As another example, the XR system may detect movement of the electronic device presenting the XR environment (e.g., a mobile phone, a tablet, a laptop, or the like) and, in response, adjust graphical content and an acoustic field presented to the person in a manner similar to how such views and sounds would change in a physical environment. In some situations (e.g., for accessibility reasons), the XR system may adjust the characteristic(s) of graphical content in the XR environment in response to representations of physical motions (e.g., vocal commands).

There are many different types of electronic systems that enable a person to sense and/or interact with various XR environments. Examples include: head mountable systems, projection-based systems, heads-up displays (HUDs), vehicle windshields having integrated display capability, windows having integrated display capability, displays formed as lenses designed to be placed on a person's eyes (e.g., similar to contact lenses), headphones/earphones, speaker arrays, input systems (e.g., wearable or handheld controllers with or without haptic feedback), smartphones, tablets, and desktop/laptop computers. A head mountable system may have one or more speaker(s) and an integrated opaque display. Alternatively, a head mountable system may be configured to accept an external opaque display (e.g., a smartphone). The head mountable system may incorporate one or more imaging sensors to capture images or video of the physical environment and/or one or more microphones to capture audio of the physical environment. Rather than an opaque display, a head mountable system may have a transparent or translucent display. The transparent or translucent display may have a medium through which light representative of images is directed to a person's eyes. The display may utilize digital light projection, OLEDs, LEDs, uLEDs, liquid crystal on silicon, laser scanning light source, or any combination of these technologies. The medium may be an optical waveguide, a hologram medium, an optical combiner, an optical reflector, or any combination thereof. In some implementations, the transparent or translucent display may be configured to become opaque selectively. Projection-based systems may employ retinal projection technology that projects graphical images onto a person's retina. Projection systems also may be configured to project virtual objects into the physical environment, for example, as a hologram or on a physical surface.

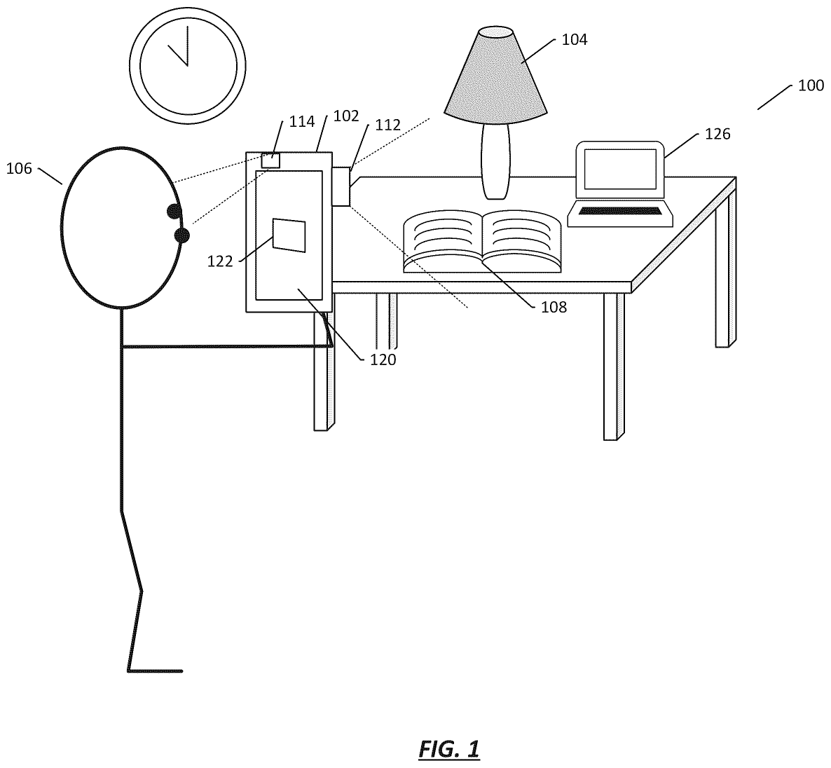

Turning to FIG. 1, an example environment diagram is presented, in accordance with one or more embodiments. According to some embodiments, a user 106 may view an environment 100 through a device 102. The device 102 may include a see-through display 120, by which the user 106 can see objects in the environment, as shown at 122. Note that although the view of the environment 122 is shown as a simple rectangle for simplicity purposes, it should be understood that in some embodiments, the view 122 of the environment 100 will actually just appear as the various objects in the environment 100.

In some embodiments, the device 102 may include various sensors by which sensor data for the environment and/or the user can be obtained, for example, one or more user-facing sensors 114 from which user data (such as eye and/or gaze information, pupil dilation, redness of sclera, tear detection and blinking rate) may be obtained. Thus, the one or more user-facing sensors may include, for example, a camera, eye tracking sensor, and/or related sensors.

The device 102 may include additional sensors from which environmental data may be obtained. The additional sensors 112 may be facing any direction. For example, the additional sensors 112 may face away from the user and/or may face toward the user in a direction orthogonal to the user, such as downward facing sensors, upward facing sensors, and the like. Further, in some embodiments, the additional sensors may be embedded within the device 102. The sensors may be embedded in the device and may include sensors such as cameras, ambient light sensors, motion detection sensors such as inertial detection sensors, microphones, flicker sensors, and the like. In some embodiments, the device 102 may determine state information for one or more components or apps of the device to determine context information. This may include, for example, Wi-Fi signals, Bluetooth connections, pairing signals, and the like.

According to one or more embodiments, the device 102 may communicably couple to one or more remote devices in the environment and may obtain additional environmental data from the remote devices. The remote devices may include, for example, additional electronic devices 126, laptops, desktop computers, Internet of Things (IoT) devices, such as thermostats, smart lighting 104, and other devices. As such, in some embodiments, the device 102 can aggregate environmental data and/or user data and infer a user activity. The user activity may include an action or performance by the user and/or may include a user state, such as if the user is in an active state or not.

The device 102 may use aggregated sensor data and other environmental data to determine a current user activity. In the example shown, the user 106 is shown reading a book 108 on a desk. Data which may be used to determine the user activity may include, for example, image data that is determined to include the book, gaze information showing the user is looking at the book, eye tracking showing the user's eyes are following the text of the book, and the like. The user activity may be an activity the user is participating in that involves using the device, for example, a head mounted device worn by the user. In some embodiments, the activity may not involve using the device having the sensors from which the sensor data is collected to determine the activity.

In some embodiments, determining the user activity may include determining whether the user is awake or otherwise active. For example, data from a wearable device such as a sleep tracker, heartbeat tracker, IMU, and the like, may provide biometric data from which a wake status may be determined. As another example, eye status (i.e., if eyes are moving or open) and head status (e.g., if the head is moving) may be used to determine a wake status.

In some embodiments, the system may determine, based on the eye characteristics, whether the eye is in a strained state or otherwise compromised. In one or more embodiments, if the eye characteristics satisfy an eye state criterion, then the device 102 may cause some mitigating or corrective action to be performed. In some embodiments, the eye state criterion may be based on environmental data, such as a lighting or other characteristic of the environment, and/or user data, such as an eye characteristic, a user activity, or the like. The corrective operation may be performed by the device 102, such as by activating a hardware and/or software component of the device. Additionally, or alternatively, the corrective operation may be performed by another device. For example, device 102 may transmit an instruction to cause the lamp 104 to be turned on or to cause the computer 126 to perform an operation.

In other embodiments, an upcoming activity may be predicted which is known to cause eye strain. For example, if a user reads every night at 10:00, the device can determine, based on the environmental data, whether the environment is predicted to cause an eye strain event or otherwise have an impact on an eye state. As such, the device 102 may preemptively cause an operation to be performed to modify an environment to avoid a user performing an activity in a context that would have an impact on an eye state.

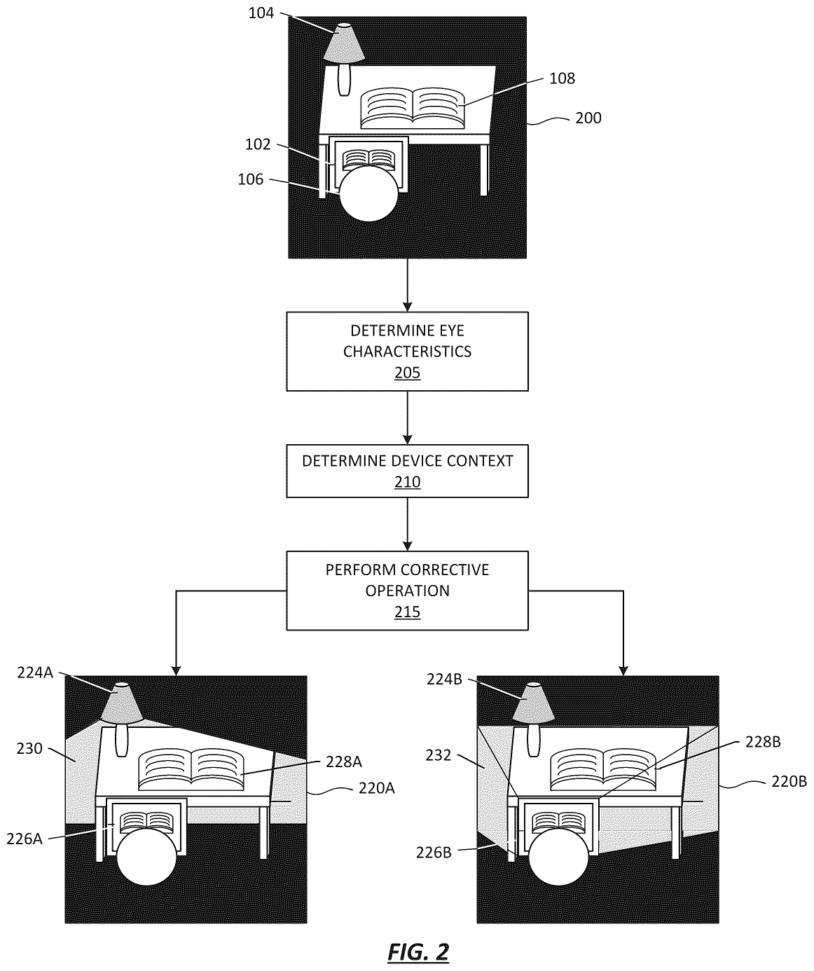

Turning now to FIG. 2, a flow diagram is presented of a technique for managing an operation of a device to address an eye state, according to one or more embodiments. The flow diagram begins at 200, showing an alternate view of the environment 100 of FIG. 1. As such, 200 shows that the user 106 is reading the book 108 in a dark room. The user 106 is reading the book via device 102, which either has a pass-through or see-through display such that the book 108 is visible to the user through the device 102.

The flow diagram continues at block 205 where eye characteristics are determined. As described above, the eye characteristics may be based on sensor data from the device 102, such as eye tracking data, gaze information, pupil dilation, color of sclera, tear detection and blinking rate. In some embodiments, the eye characteristics may also include eye information from which a user activity can be determined and/or inferred. For example, in scene 200, gaze, vergence, and eye motion may be tracked and determined to be compatible with a “reading” activity. For example, the user 106 may have their eyes open, and their gaze may be moving in a pattern corresponding to reading, with a vergence of 0.2-1 meters, for a certain duration. In some embodiments, the eye characteristics may be used to compare the eye data to an eye state criterion. For example, the eye state criterion may indicate eye state characteristics associated with eye strain such that eye strain in the user can be detected based on the collected sensor data.

According to some embodiments, the eye state criterion may be based on a device context, as shown at block 210. Thus, in some embodiments, sensor data and other environmental data may be aggregated by the device to determine a current context. This may include, for example, ambient lighting, screen brightness, time of day, noise level, objects in the scene, and the like. As an example, in the scene 200, it may be determined, based on object detection, that a book 108 is the focus of the user's 106 attention. Further, in some embodiments, higher-level information may be used to determine context and may be obtained from operation of the local device and/or network devices. This higher-level information includes, for example, time of day, weather, and semantics, such as whether a user is indoors or outdoors, a particular room in which the user is located, and the like. Thus, it may be determined (based on data such as local time, weather information, and the like) that the user is likely in the dark. The device context may also be used to determine the user activity. For example, an electronic reading device may be determined based on Bluetooth pairings or the like. As another example, the device may detect hand motion, such as a hand performing a turn of a physical page, that is consistent with a reading activity.

The flowchart continues to block 215 where, based on the eye characteristics and/or device context, a corrective operation is performed. As described above, the corrective operation may be performed by the local device and/or by directing one or more additional devices to perform the operation. The corrective operation may change characteristics of the environment and/or the user activity such that the eye state criterion is no longer satisfied. For example, returning to 200 above, if it is determined that the user is reading a book in the dark, then the corrective operation may be to increase the lighting in the environment. In some embodiments, as shown at 220A, the device 226A may cause a lamp 224A to be activated or turned on such that the book 228A is lit 230 in the environment. For example, lamp 224A may be part of a connected “smart home” network which can be operated from the device 226A. As another example, as shown at 220B, the device 226B may engage an external-facing light 232 to light the book 228B, such that the book 228B is lit in the environment while the lamp 224B remains turned off. As such, the user 226 can read the book 228 in the darkened room in a lit environment to reduce eye strain.

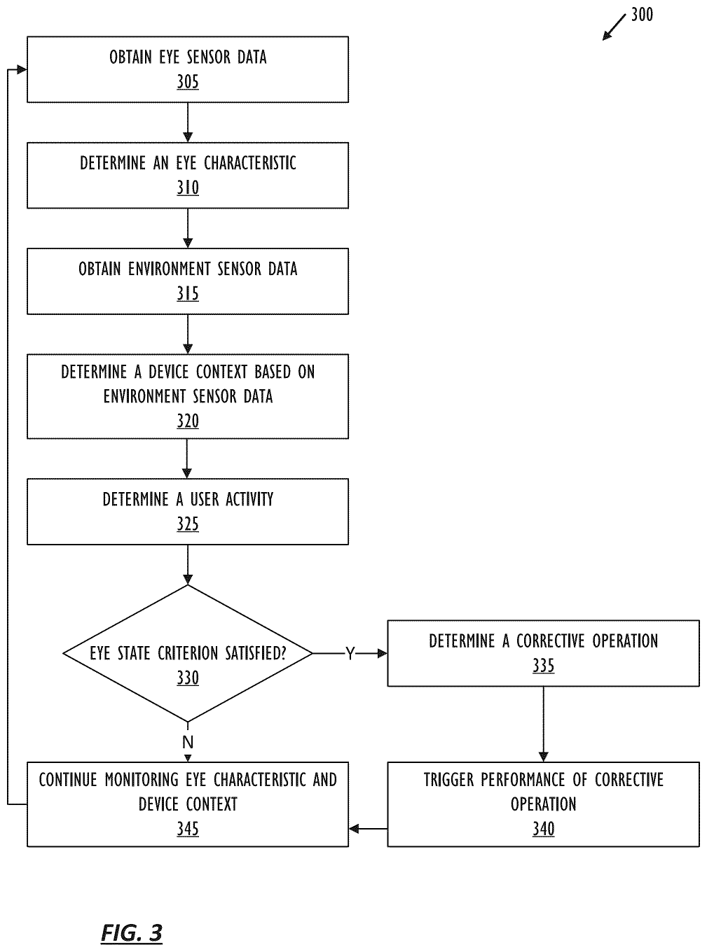

FIG. 3 shows, in flowchart form, an example process for selecting and triggering performance of a corrective operation, in accordance with one or more embodiments. For purposes of explanation, the following steps will be described in the context of FIG. 1. However, it should be understood that the various actions may be taken by alternate components. In addition, the various actions may be performed in a different order. Further, some actions may be performed simultaneously, and some may not be required, or others may be added, according to various embodiments.

The flowchart 300 begins at block 305 where the device obtains eye sensor data. The eye sensor data may be captured by one or more sensors that are part of a wearable device, such as a head mounted device, which the user may be wearing as they are experiencing a physical environment in their surroundings. The eye sensor data obtained at 305 (including image data, eye tracking data, and the like) may be collected by one or more sensors.

At block 310, one or more eye characteristics are determined based on the eye sensor data. The eye characteristics may include, for example, whether an eye is open or closed, a redness level, a blink rate, a dilation measurement, and the like. In some embodiments, the one or more eye characteristics may additionally or alternatively be related to a user activity. For example, a user activity may be determined based on the collected eye sensor data. As an example, a user can be determined to be asleep or awake (or otherwise in an active state). Further, the user can be determined to be engaging in a particular activity based on eye characteristics. As an example, based on gaze, vergence, and eye motion, a user's eye characteristics may be determined to be compatible with a “watching a screen” activity.

The flowchart continues at block 315, and environment sensor data is obtained. The environment sensor data may include data from sensors on the local device or be obtained from sensors on remote devices, in some embodiments. The sensors may be embedded in the device and may include sensors, such as cameras, ambient light sensors, motion detection sensors such as inertial detection sensors, microphones, flicker sensors, and the like. In some embodiments, the device 102 may determine state information for one or more components or apps of the device in order to determine context information. This may include, for example, Wi-Fi signals, Bluetooth connections, pairing signals, and the like.

At block 320, a device context is determined based on the environment sensor data. The device context may include physical characteristics of the environment in which the device is located. For example, ambient light sensors may be used to collect data from which it may be determined whether the device is in a lit environment or a dark context. As another example, a local time and/or weather report may indicate that it is night or overcast outside, lending to an inference that the device is in a dark context.

The flowchart continues at block 325, and a user activity is determined. According to some embodiments, a local device can use the aggregated environmental data to determine or refine a user activity and/or device context. As an example scenario, from image data a device can determine, based on depth data, that a screen is present in the environment at a particular distance. Flicker sensor data may detect the flickering of a screen to determine that the screen is on. A microphone may be used to collect audio data from which audio details can be determined, such as media identification, a location of a source of the sound, and the like. Motion data from an IMU may indicate that a user's head is mostly in a static position. The combination of this sensor data may be determined to be consistent with a “watching a screen” activity. For example, a user using the device may be watching a television in the physical environment.

At block 330, a determination is made regarding whether an eye state criterion is satisfied. According to some embodiments, the eye state criterion may be based on eye characteristics that are consistent with a predetermined eye condition, such as eye strain. In some embodiments, the eye state criterion may additionally or alternatively be dependent upon a user activity and/or device context. For example, in some embodiments, if the eye characteristics do not indicate a predetermined condition, the eye state criterion may still be satisfied, for example, if a known user activity, device context, or combination thereof is determined to be linked to a predetermined eye condition, such as reading in the dark, focusing on a near object for a long period of time, and the like.

If at block 330 it is determined that an eye state criterion is satisfied, then the flowchart 300 continues to block 335, and the device determines a corrective operation. According to one or more embodiments, the corrective operation may be an operation performed by the local device and/or a remote device, which, in turn, resolves a condition in the environment and/or user activity such that the predetermined condition is resolved or lessened. In the example above, if it is determined that the user is watching a screen and that the environment is dark, then a corrective operation may include activating a blue light filter on the local device, for example, if the local device includes a see-through display such that external blue light is blocked from entering the user's eye. As another example, the corrective operation may include triggering another device to perform an operation, such as triggering the device comprising the screen being watched by the user to adjust luminosity display configuration, color temperature, and the like. Thus, at block 340, the device triggers performance of the corrective operation, and the flowchart proceeds to block 340, where the device continues monitoring the eye characteristics and device context. Similarly, returning to block 330, if it is determined that the eye state criterion is not satisfied, then the flowchart also continues to block 345, and the device continues monitoring the eye characteristics and device context.

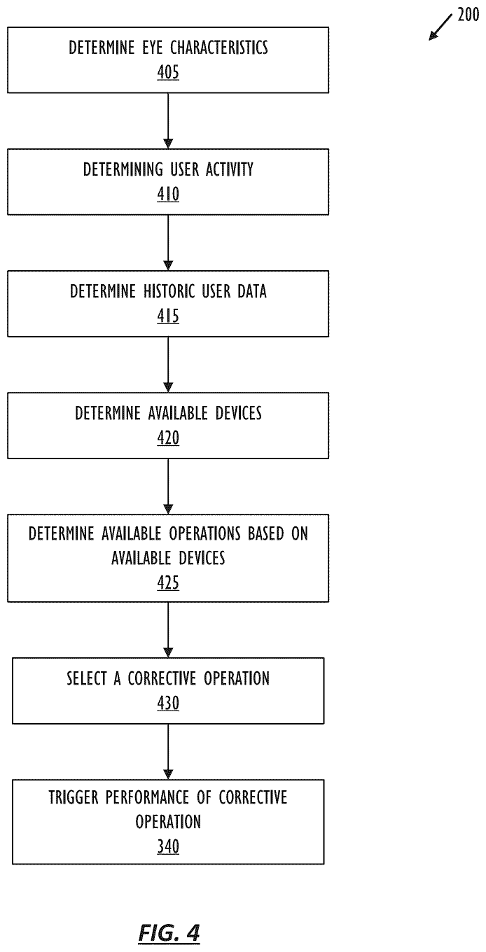

As described above, in some embodiments, a corrective operation may be performed based on temporal or historic information. Thus, FIG. 4 shows, in flowchart form, an example process for selecting and triggering performance of a corrective operation, in accordance with one or more embodiments. For purposes of explanation, the following steps will be described in the context of FIG. 3. However, it should be understood that the various actions may be taken by alternate components. In addition, the various actions may be performed in a different order. Further, some actions may be performed simultaneously, and some may not be required, or others may be added, according to various embodiments.

The flowchart 400 begins at block 405 where the device determines eye characteristics. The eye characteristics may include, for example, whether an eye is open or closed, a redness level, a blink rate, a dilation measurement, and the like. In some embodiments, the one or more eye characteristics may additionally or alternatively be related to a user activity. For example, a user activity may be determined based on the collected eye sensor data. As an example, a user can be determined to be asleep or awake (or otherwise in an active state). Further, the user can be determined to be engaging in a particular activity based on eye characteristics. As an example, based on gaze, vergence, and eye motion, a user's eye characteristics may be determined to be compatible with a “watching a screen” activity, a “reading a book” activity, or the like.

At block 410, user activity is determined. The user activity may or may not involve the device determining the user activity. For example, as shown in FIG. 1, the activity includes reading a physical book, which happens to be visible through the device 102. Other examples of activities include staring at something at a particular distance for a threshold amount of time. As an example, viewing something up close, such as while knitting or crocheting without taking a break or driving for a certain period of time. Another example may include performing an activity in a dark environment, such as reading a book, watching a screen, or other activities which require a user's eyes to strain in the dark. Yet other examples include a user being active in certain climate conditions, such as an environment with a threshold UV index, humidity, and the like.

In some embodiments, historic data may be considered in determining a corrective operation or refining a corrective operation, as shown at block 415. As an example, the device can determine that the user has spent a certain amount of time in a dark environment for the day, a certain amount of time looking at blue light after sunset, and the like. As another example, a user's historic information may be tracked from which the device may determine a routine based on patterns in the user's data. For example, the device may determine that a user routinely reads an e-book on a secondary electronic device for 30 minutes before turning out the lights and going to sleep.

At block 420, a determination is made regarding whether additional devices are available to perform corrective operations. The determination may be made, for example, based on identifying accessory devices connected to the local device, secondary devices located on a same network, IoT devices communicably coupled to the local device, and the like. Then, at block 425, available corrective operations are determined based on capabilities of the local device and/or additional devices communicably coupled to the local device.

The flowchart continues at block 430 where a corrective operation is selected. The corrective operation may be selected from among the available operations determined at block 425. Further, in some embodiments, the corrective operation may be selected based on system resources. As an example, if the local device is determined to have multiple potential corrective operations available to address a particular activity, and the local device has limited power available, a low power solution may be selected. As another example, user data may be used to select a corrective operation. As an example, a user may not wish to turn on any additional lights in an environment but may approve reducing a brightness of a screen. Those user-defined parameters may be determined, for example, from a user profile.

Further, a combination of the various determinations made above with respect to blocks 405-425 may be used to select a corrective operation. For example, if the local device determines the user's eye redness is greater than some threshold after 27 minutes of reading, and the user typically reads for 30 minutes, the device may respond with more minor adjustments to the operating parameters than if the user routinely reads for an hour before going to sleep and has another 33 minutes of reading to go.

The flowchart 400 concludes at block 340, as shown in FIG. 3, and the device triggers performance of the corrective operation. In some embodiments, as shown in FIG. 3, the flowchart can proceed to block 345 where the device continues monitoring the eye characteristics and device context.

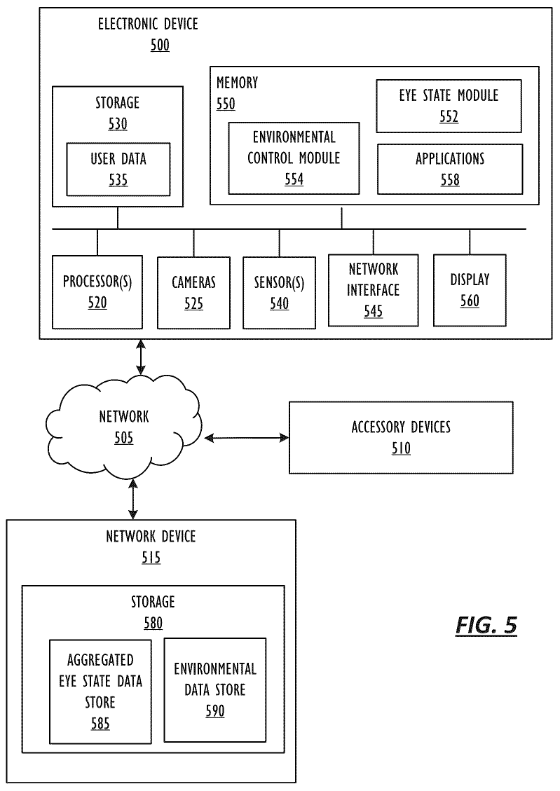

FIG. 5 depicts a network diagram for a system by which various embodiments of the disclosure may be practiced. Specifically, FIG. 5 depicts an electronic device 500 that is a computer system. Electronic device 500 may be part of a multifunctional device, such as a mobile phone, tablet computer, personal digital assistant, portable music/video player, wearable device, head-mounted systems, projection-based systems, base station, laptop computer, desktop computer, network device, or any other electronic system such as those described herein. Electronic device 500 may be connected to other devices across a network 505, such as an accessory electronic device 510, mobile devices, tablet devices, desktop devices, and remote sensing devices, as well as network device 515. Accessory devices 510 may include, for example, additional laptop computers, desktop computers, mobile devices, wearable devices and other devices communicably coupled to electronic device 500. In some embodiments, accessory devices 510 may include IoT devices communicably coupled to the electronic device 500 and having one or more sensors by which environmental data can be captured and/or by which one or more corrective operations may be performed. Network device 515 may be any kind of electronic device communicably coupled to electronic device 500 across network 505 via network interface 545. In some embodiments, network device 515 may include network storage, such as cloud storage and the like. Network 505 may include one or more types of networks across which the various electronic components are communicably coupled. Illustrative networks include, but are not limited to, a local network, such as a universal serial bus (USB) network, an organization's local area network, and a wide area network, such as the Internet.

Electronic device 500, accessory electronic devices 510, and/or network device 515 may additionally or alternatively include one or more additional devices within which the various functionality may be contained or across which the various functionality may be distributed, such as server devices, base stations, accessory devices, and the like. It should be understood that the various components and functionality within electronic device 500, additional electronic device 510, and network device 515 may be differently distributed across the devices or may be distributed across additional devices.

Electronic device 500 may include a processor 520. Processor 520 may be a system-on-chip, such as those found in mobile devices, and include one or more central processing units (CPUs), dedicated graphics processing units (GPUs), or both. Further, processor 520 may include multiple processors of the same or different type. Electronic device 500 may also include a memory 550. Memory 550 may include one or more different types of memory which may be used for performing device functions in conjunction with processor 520. For example, memory 550 may include cache, ROM, RAM, or any kind of transitory or non-transitory computer readable storage medium capable of storing computer readable code. Memory 550 may store various programming modules during execution, such as eye state module 552 which is configured to determine one or more of user activity, device context, and/or eye characteristics, and in addition, the eye state module 552 may determine whether an eye state criterion is satisfied therefrom. In some embodiments, eye state module 552 may determine a corrective operation to be performed by an electronic device and/or another device, such as one or more of accessory devices 510 and network devices 515, and trigger such corrective operation to be performed. Memory 550 also includes an environmental control module 554 which is configured to perform such corrective operation and/or transmit an instruction to one or more secondary devices (e.g., accessory devices 510, network device 515, and the like) to perform the corrective operation. Further, memory 550 may include one or more additional applications 558. In some embodiments, a state of the applications 558 may be used by the eye state module 552 to determine user activity, device context, and the like.

Electronic device 500 may also include storage 530. Storage 530 may include one or more non-transitory computer-readable mediums including, for example, magnetic disks (fixed, floppy, and removable) and tape, optical media such as CD-ROMs and digital video disks (DVDs), and semiconductor memory devices such as Electrically Programmable Read-Only Memory (EPROM) and Electrically Erasable Programmable Read-Only Memory (EEPROM). Storage 530 may be utilized to store various data and structures which may be utilized for mitigating triggering conditions in a pass-through environment. For example, storage 530 may include a user data store 535 which may include user-configurable data related to the eye state correction operations. For example, the user data 535 may include user profile data indicating user preference for selecting a corrective operation. In some embodiments, the eye state module 552 may infer user habits for predictive determination of device context, user activity, and the like. As such, user data 535 may include data related to user history and predictions related to user habits.

Electronic device 500 may include a set of sensors 540. In this example, the set of sensors 540 may include one or more image capture sensors, an ambient light sensor, a motion sensor, an eye tracking sensor, and the like. In other implementations, the set of sensors 540 further includes an accelerometer, a global positioning system (GPS), a pressure sensor, and the inertial measurement unit (IMU), and the like.

Electronic device 500 may allow a user to interact with XR environments. Many electronic systems enable an individual to interact with and/or sense various XR settings. One example includes head mounted systems. A head mounted system may have an opaque display and speaker(s). Alternatively, a head mounted system may be designed to receive an external display (e.g., a smartphone). The head mounted system may have imaging sensor(s) and/or microphones for taking images/video and/or capturing audio of the physical setting, respectively. A head mounted system also may have a transparent or semi-transparent see-through display 560. The transparent or semi-transparent display may incorporate a substrate through which light representative of images is directed to an individual's eyes. The display may incorporate LEDs, OLEDs, a digital light projector, a laser scanning light source, liquid crystal on silicon, or any combination of these technologies. The substrate through which the light is transmitted may be a light waveguide, optical combiner, optical reflector, holographic substrate, or any combination of these substrates. In one embodiment, the transparent or semi-transparent display may transition selectively between an opaque state and a transparent or semi-transparent state. In another example, the electronic system may be a projection-based system. A projection-based system may use retinal projection to project images onto an individual's retina. Alternatively, a projection system also may project virtual objects into a physical setting (e.g., onto a physical surface or as a holograph). Other examples of XR systems include heads up displays, automotive windshields with the ability to display graphics, windows with the ability to display graphics, lenses with the ability to display graphics, headphones or earphones, speaker arrangements, input mechanisms (e.g., controllers having or not having haptic feedback), tablets, smartphones, and desktop or laptop computers.

In some embodiments, network device 515 includes storage 580. Storage 580 may include one or more storage devices and may be located on one or more server devices. According to one or more embodiments, storage 580 includes data related to eye state determination and corrective operations. For example, storage 580 may include aggregated eye state data store 585. In some embodiments, the aggregated eye state data may be used to store anonymized data related to environmental conditions that are associated with predetermined eye conditions, such as eye strain. In some embodiments, the aggregated eye state data store may be used to determine eye state criteria which should be considered for determining whether to cause a corrective operation to be performed. The storage 580 may also include an environmental data store 590. The environmental data store may include data from remote servers that affect the environment, such as weather, pollution levels, and the like.

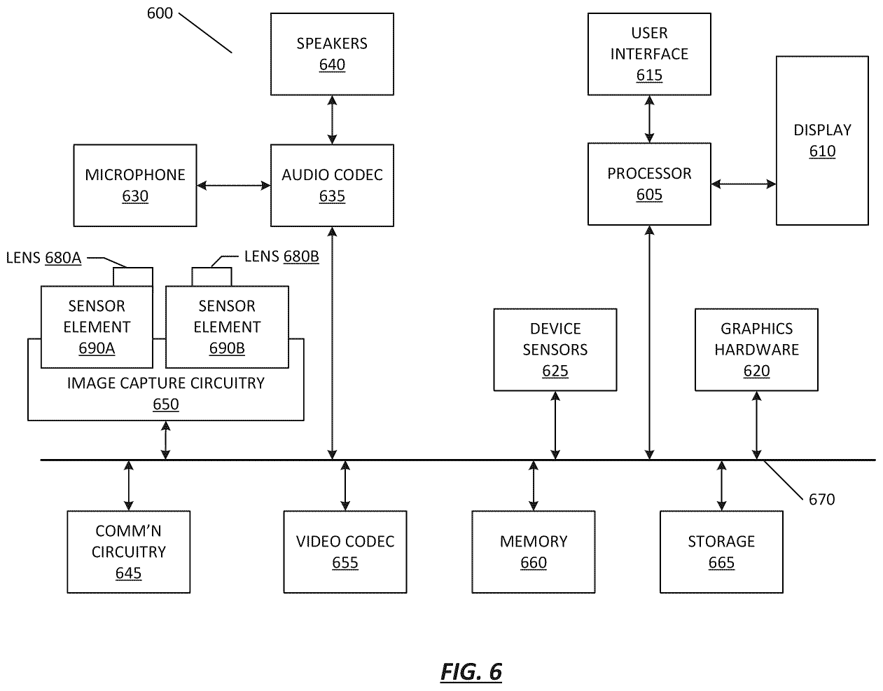

Referring now to FIG. 6, a simplified functional block diagram of illustrative multifunction electronic device 600 is shown according to one embodiment. Each of electronic devices may be a multifunctional electronic device or may have some or all of the components of a multifunctional electronic device described herein. Multifunction electronic device 600 may include some combination of processor 605, display 610, user interface 615, graphics hardware 620, device sensors 625 (e.g., proximity sensor/ambient light sensor, accelerometer and/or gyroscope), microphone 630, audio codec 635, speaker(s) 640, communications circuitry 645, digital image capture circuitry 650 (e.g., including camera system), memory 660, storage device 665, and communications bus 670. Multifunction electronic device 600 may be, for example, a mobile telephone, personal music player, wearable device, tablet computer, or the like.

Processor 605 may execute instructions necessary to carry out or control the operation of many functions performed by device 600. Processor 605 may, for instance, drive display 610 and receive user input from user interface 615. User interface 615 may allow a user to interact with device 600. For example, user interface 615 can take a variety of forms, such as a button, keypad, dial, a click wheel, keyboard, display screen, touch screen, and the like. Processor 605 may also, for example, be a system-on-chip, such as those found in mobile devices, and include a dedicated graphics processing unit (GPU). Processor 605 may be based on reduced instruction-set computer (RISC) or complex instruction-set computer (CISC) architectures or any other suitable architecture and may include one or more processing cores. Graphics hardware 620 may be special purpose computational hardware for processing graphics and/or assisting processor 605 to process graphics information. In one embodiment, graphics hardware 620 may include a programmable GPU.

Image capture circuitry 650 may include one or more lens assemblies, such as 680A and 680B. The lens assemblies may have a combination of various characteristics, such as differing focal length and the like. For example, lens assembly 680A may have a short focal length relative to the focal length of lens assembly 680B. Each lens assembly may have a separate associated sensor element 690. Alternatively, two or more lens assemblies may share a common sensor element. Image capture circuitry 650 may capture still images, video images, enhanced images, and the like. Output from image capture circuitry 650 may be processed, at least in part, by video codec(s) 655, processor 605, graphics hardware 620, and/or a dedicated image processing unit or pipeline incorporated within circuitry 645. Images so captured may be stored in memory 660 and/or storage 665.

Memory 660 may include one or more different types of media used by processor 605 and graphics hardware 620 to perform device functions. For example, memory 660 may include memory cache, read-only memory (ROM), and/or random-access memory (RAM). Storage 665 may store media (e.g., audio, image, and video files), computer program instructions or software, preference information, device profile information, and any other suitable data. Storage 665 may include one more non-transitory computer-readable storage mediums, including, for example, magnetic disks (fixed, floppy, and removable) and tape, optical media such as CD-ROMs and digital video disks (DVDs), and semiconductor memory devices such as Electrically Programmable Read-Only Memory (EPROM) and Electrically Erasable Programmable Read-Only Memory (EEPROM). Memory 660 and storage 665 may be used to tangibly retain computer program instructions or computer readable code organized into one or more modules and written in any desired computer programming language. When executed by, for example, processor 605, such computer program code may implement one or more of the methods described herein.

It is to be understood that the above description is intended to be illustrative and not restrictive. The material has been presented to enable any person skilled in the art to make and use the disclosed subject matter as claimed and is provided in the context of particular embodiments, variations of which will be readily apparent to those skilled in the art (e.g., some of the disclosed embodiments may be used in combination with each other). Accordingly, the specific arrangement of steps or actions shown in FIGS. 3-4, or the arrangement of elements shown in FIGS. 1-2 and 5-6, should not be construed as limiting the scope of the disclosed subject matter. The scope of the invention, therefore, should be determined with reference to the appended claims, along with the full scope of equivalents to which such claims are entitled. In the appended claims, the terms “including” and “in which” are used as the plain English equivalents of the respective terms “comprising” and “wherein.”

The techniques defined herein consider the option of obtaining and utilizing a user's personal information. For example, such personal information may be utilized in order to provide a multi-user communication session on an electronic device. However, to the extent such personal information is collected, such information should be obtained with the user's informed consent such that the user has knowledge of and control over the use of their personal information.

Parties having access to personal information will utilize the information only for legitimate and reasonable purposes and will adhere to privacy policies and practices that are at least in accordance with appropriate laws and regulations. In addition, such policies are to be well-established, user-accessible, and recognized as meeting or exceeding governmental/industry standards. Moreover, the personal information will not be distributed, sold, or otherwise shared outside of any reasonable and legitimate purposes.

Users may, however, limit the degree to which such parties may obtain personal information. The processes and devices described herein may allow settings or other preferences to be altered such that users control access of their personal information. Furthermore, while some features defined herein are described in the context of using personal information, various aspects of these features can be implemented without the need to use such information. As an example, a user's personal information may be obscured or otherwise generalized such that the information does not identify the specific user from which the information was obtained.

1. A method comprising:
obtaining environment sensor data from a first one or more sensors of a device;
determining a current context for the device based on the environment sensor data;
determining that an eye state criterion is satisfied based on the current context;
in response to determining that the eye state criterion is satisfied, determining a corrective operation in accordance with the eye state criterion; and
causing the corrective operation to be performed, wherein performance of the corrective operation adjusts an environmental condition associated with the eye state criterion.

2. The method of claim 1, wherein the eye characteristic comprises at least one of eye coloration, pupil dilation, and blinking rate.

3. The method of claim 1, wherein the current context comprises at least one of ambient light, a current device activity, and a user routine.

4. The method of claim 1, wherein causing the corrective operation to be performed comprises transmitting a triggering notification to a second device to perform the corrective operation.

5. The method of claim 1, wherein causing the corrective operation to be performed comprises modifying an operation of the device to perform the corrective operation.

6. The method of claim 1, further comprising:
determining a user activity based on sensor data from at least one of the first on or more sensors or a second sensor, wherein the eye characteristic is determined in accordance with the user activity.

7. The method of claim 1, further comprising:
obtaining additional environmental data from a second device, wherein the current context for the device is further determined based on the additional environmental data.

8. A non-transitory computer readable medium comprising computer readable code executable by one or more processors to:
obtain environment sensor data from a first one or more sensors of a device;
determine a current context for the device based on the environment sensor data;
determine that an eye state criterion is satisfied based on the current context;
in response to determining that the eye state criterion is satisfied, determine a corrective operation in accordance with the eye state criterion; and
cause the corrective operation to be performed, wherein performance of the corrective operation adjusts an environmental condition associated with the eye state criterion.

9. The non-transitory computer readable medium of claim 8, wherein the eye characteristic comprises at least one of eye coloration, pupil dilation, and blinking rate.

10. The non-transitory computer readable medium of claim 8, wherein the current context comprises at least one of ambient light, a current device activity, and a user routine.

11. The non-transitory computer readable medium of claim 8, wherein the computer readable code to cause the corrective operation to be performed comprises computer readable code to transmit a triggering notification to a second device to perform the corrective operation.

12. The non-transitory computer readable medium of claim 8, wherein the computer readable code to cause the corrective operation to be performed comprises computer readable code to modify an operation of the device to perform the corrective operation.

13. The non-transitory computer readable medium of claim 8, further comprising computer readable code to:
determine a user activity based on sensor data from at least one of the first on or more sensors or a second sensor, wherein the eye characteristic is determined in accordance with the user activity.

14. The non-transitory computer readable medium of claim 8, further comprising computer readable code to:
obtain additional environmental data from a second device, wherein the current context for the device is further determined based on the additional environmental data.

15. A system comprising:
one or more processors; and
one or more non-transitory computer readable media comprising computer readable code executable by the one or more processors to:
obtain environment sensor data from a first one or more sensors of a device;
determine a current context for the device based on the environment sensor data;
determine that an eye state criterion is satisfied based on the current context;
in response to determining that the eye state criterion is satisfied, determine a corrective operation in accordance with the eye state criterion; and
cause the corrective operation to be performed, wherein performance of the corrective operation adjusts an environmental condition associated with the eye state criterion.

16. The system of claim 15, wherein the eye characteristic comprises at least one of eye coloration, pupil dilation, and blinking rate.

17. The system of claim 15, wherein the current context comprises at least one of ambient light, a current device activity, and a user routine.

18. The system of claim 15, wherein the computer readable code to cause the corrective operation to be performed comprises computer readable code to transmit a triggering notification to a second device to perform the corrective operation.

19. The system of claim 15, wherein the computer readable code to cause the corrective operation to be performed comprises computer readable code to modify an operation of the device to perform the corrective operation.

20. The system of claim 15, further comprising computer readable code to:
determine a user activity based on sensor data from at least one of the first on or more sensors or a second sensor, wherein the eye characteristic is determined in accordance with the user activity.
